(19) United States
(12) Patent Application Publication
(10) Pub. No.: US 2024/0062864 A1
(43) Pub. Date: Feb. 22, 2024

(54) TEMPORAL DATA AUGMENTATION AND PREDICTION USING MULTI-STAGE MACHINE-LEARNING BASED MODELS

(71) Applicant: UnitedHealth Group Incorporated, Minnetonka, MN (US)

(72) Inventors: Eran HALPERIN, Santa Monica, CA (US); Gregory L. LYNG, Minneapolis, MN (US); Brian L. HILL, Culver City, CA (US)

(21) Appl. No.: 18/057,785

(22) Filed: Nov. 22, 2022

Related U.S. Application Data
(60) Provisional application No. 63/371,947, filed on Aug. 19, 2022.

(51) Int. Cl. G16H 20/10

(52) U.S. Cl. CPC G16H 20/10

(57) ABSTRACT

Various embodiments of the present disclosure disclose machine-learning based data augmentation and prediction techniques for generating predictive classifications based on temporal data. A machine-learning based model is provided that can receive an input data object associated with a plurality of predictive temporal parameters; determine augmented temporal data objects based on the predictive temporal parameters; generate predictive data representations for the input data object based on the predictive temporal parameters and the augmented temporal data objects; generate a multi-channel predictive data representation based on the predictive data representations for the input data object; and generate a predictive classification for the input data object based on the multi-channel predictive data representation.

### CROSS-REFERENCE TO RELATED APPLICATIONS

This application claims the benefit of U.S. Provisional Application No. 63/371,947, entitled “MULTI-CHANNEL TEMPORAL CLASSIFICATION IN CONTINUOUS GLUCOSE MONITOR (CGM) DATA,” and filed Aug. 19, 2022, the entire contents of which are hereby incorporated by reference.

### BACKGROUND

Various embodiments of the present disclosure address technical challenges related to machine-learning based predictive models and predictive data modeling given limitations of existing predictive data analysis processes. By way of example, due to an inability to accurately process or interpret primary factors such as time-based physiological signals for certain predictive insights, existing predictive data analysis processes make inferences based on secondary factors that may be weakly or inaccurately correlated to a predictive insight. Such techniques can be inaccurate, time-consuming, and unreliable. Various embodiments of the present disclosure make important contributions to various existing predictive data analysis processes by facilitating the accurate interpretation of primary factors for predictive insights.

### BRIEF SUMMARY

Various embodiments of the present disclosure describe machine-learning based data augmentation and prediction techniques for generating predictive classifications based on temporal data. In some embodiments, a machine-learning based temporal classification model can facilitate a multi-stage temporal data processing scheme that can augment temporal data and process the augmented temporal data with a machine-learning based model to generate a predictive classification. Using some of the techniques described herein, a proposed system can augment and process temporal data to overcome the limitations of existing machine-learning based prediction techniques.

In one implementation, a computer-implemented method for machine-learning based input data augmentation and analysis is provided. The method comprises determining, using one or more processors, one or more augmented temporal data objects based on a plurality of predictive temporal parameters of an input data object associated with a predictive classification process, wherein the augmented temporal data objects comprise at least one transformation of the predictive temporal parameters; generating, using the processors, one or more predictive data representations for the input data object based on the predictive temporal parameters and the augmented temporal data objects; generating, using the processors, a multi-channel predictive data representation based on the predictive data representations for the input data object; and generating, using the processors and a machine-learning based input data object classification model, a predictive classification for the input data object based on the multi-channel predictive data representation.

In another implementation, an apparatus for machine-learning based input data augmentation and analysis is provided. The apparatus comprises at least one processor and at least one memory including program code. The at least one memory and the program code are configured to, with the at least one processor, cause the apparatus to at least determine one or more augmented temporal data objects based on a plurality of predictive temporal parameters of an input data object associated with a predictive classification process, wherein the augmented temporal data objects comprise at least one transformation of the predictive temporal parameters; generate one or more predictive data representations for the input data object based on the predictive temporal parameters and the augmented temporal data objects; generate a multi-channel predictive data representation based on the predictive data representations for the input data object; and generate, using a machine-learning based input data object classification model, a predictive classification for the input data object based on the multi-channel predictive data representation.

In yet another implementation, a computer program product for machine-learning based input data augmentation and analysis is provided. The computer program product comprises at least one non-transitory computer-readable storage medium having computer-readable program code portions stored therein. The computer-readable program code portions configured to determine one or more augmented temporal data objects based on a plurality of predictive temporal parameters of an input data object associated with a predictive classification process, wherein the augmented temporal data objects comprise at least one transformation of the predictive temporal parameters; generate one or more predictive data representations for the input data object based on the predictive temporal parameters and the augmented temporal data objects; generate a multi-channel predictive data representation based on the predictive data representations for the input data object; and generate, using a machine-learning based input data object classification model, a predictive classification for the input data object based on the multi-channel predictive data representation.

### BRIEF DESCRIPTION OF THE DRAWINGS

Having thus described the invention in general terms, reference will now be made to the accompanying drawings, which are not necessarily drawn to scale, and wherein:

FIG. 1 provides an example overview of a system that can be used to practice embodiments of the present disclosure.

FIG. 2 provides an example predictive data analysis computing entity in accordance with some embodiments discussed herein.

FIG. 3 provides an example external computing entity in accordance with some embodiments discussed herein.

FIG. 4 provides a dataflow diagram of an example predictive classification process for generating a predictive classification based on time-based data in accordance with some embodiments discussed herein.

FIG. 5 provides an operational example of a multi-channel predictive data representation in accordance with some embodiments discussed herein.

FIG. 6 is a flowchart showing an example of a process for generating a predictive classification based on time-based data in accordance with some embodiments discussed herein.

FIG. 7 is a flowchart showing an example of a process for generating a multi-channel predictive data representation in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout. Moreover, while certain embodiments of the present disclosure are described with reference to predictive data analysis, one of ordinary skill in the art will recognize that the disclosed concepts can be used to perform other types of data analysis.

### I. Overview and Technical Advantages

Embodiments of the present disclosure present new data processing and prediction techniques to improve computer interpretation of temporal data. To do so, the present disclosure describes a machine-learning based temporal classification model that is configured to perform a multi-stage predictive classification process. At a first stage, the machine-learning based temporal classification model generates a new multi-channel data structure with a plurality of temporal features from a set of time-based data. At a second stage, a machine-learning model is trained to generate a predictive classification based on the multi-channel data structure. The multi-channel data structure can be tailored to the machine-learning model by emphasizing and representing temporal features that optimize the performance of the machine-learning model. In addition, the multi-channel data structure can enable the use of different types of machine-learning techniques previously unavailable for temporal-based predictive classifications. In this way, the present disclosure provides an end-to-end predictive classification process with improved prediction accuracy relative to conventional prediction techniques.

More particularly, the machine-learning based temporal classification model can perform a plurality of data manipulation and augmentation techniques on a set of time-based data. The time-based data can include raw timeseries data measurements that are generated by digital sensors such as continuous glucose monitors (“CGM”). Each of the employed data manipulation and augmentation techniques can be tailored to the time-based data and/or a machine-learning based model used in the second stage of the multi-stage predictive classification process. For instance, each technique can be configured to extract or emphasize temporal features of the time-based data that may be relevant to a predictive classification output by a respective machine-learning based model. At times, portions of the time-based data can be segregated from the set of time-based data using contextual data to inform the intelligent selection of data relevant to the predictive classification.

The time-based data can be used to generate a plurality of channels that are each engineered to assist in improved predictive classification. One example of a channel includes a predictive data representation generated from the time-based data or derivatives/integrals of the time-based data. A predictive data representation can include a heat map that is generated using wavelet transforms that can determine a wavelet coefficient for the time-based data based on a timing and frequency of different parameters of the time-based data. The type of wavelet transform can be automatically selected to maximize the accuracy of a temporal-based predictive classification. A heat map can include an efficient three-dimensional representation of the time-based data in which the wavelet coefficient can be represented by a third dimension such as a color intensity. A plurality of predictive data representations can be grouped together to create multi-channel predictive data representation with a plurality of temporal features.

At a second stage, a machine-learning model is trained to generate a predictive classification based on the multi-channel predictive data representation. For instance, the multi-channel predictive data representation can include a multi-channel heat map image. The second stage can train a supervised image classification model with labeled sets of multi-channel predictive data representations to output accurate predictive classifications. The trained machine-learning model can be used to classify unlabeled multi-channel predictive data representations that are generated based on time-based data. The machine-learning model can leverage multiple channels of temporal information that are efficiently represented using multiple predictive data representations. At times, each channel can be weighted differently with respect to one another to improve overall performance of the machine-learning model. By using multiple, potentially differently weighted, channels, the machine-learning model can be trained to outperform predictive classifications that rely on a single channel. For instance, the performance of a three-channel model has been shown to outperform single-channel classification methods.

Example inventive and technologically advantageous embodiments of the present disclosure include: (i) new multi-channel machine-learning based image classification techniques that utilize heat maps to improve classification accuracy of temporal data; (ii) techniques for generating multiple heat maps from temporal data and derivatives of the temporal data using transformation functions such as wavelet transforms; (iii) weighting techniques for modifying the relative weight of a plurality of heat maps for a classification task; and (iv) input data generation techniques that utilize digital sensor outputs over a time window.

The new data processing and prediction techniques described herein can be applied to any set of time-based data for generating a predictive classification. In one embodiment, the multi-stage predictive classification process of the present disclosure can be applied to the problem of medical adherence and can provide a technical solution for determining whether an individual is taking medication. For example, the multi-stage predictive classification process can enable the accurate determination of such inferences based on raw digital signals that are processed to generate machine-interpretable data structures. In this way, the techniques described herein can provide a non-invasive, inexpensive, and non-time-consuming solution that can replace conventional, flawed techniques for measuring medication adherence that rely on secondary measures such as pill counts and/or self-reporting. In a medication adherence setting, the multi-stage predictive classification process can provide for a passive, easily administered technique that can accurately determine medical adherence from single physiological signals. The medical adherence setting is one example use case for the multi-stage predictive classification process described herein. The machine-learning based temporal classification model of the present disclosure provides technical improvements to both data processing and machine-learning based models that are applicable to a range of machine-learning applications including, for example, predictive models for disease progression, diagnosis, glucose dynamics, and/or other predictive use cases.

The term “input data object” can refer to a data entity that describes an initial input to a machine-learning based model. The input data object can represent a data entity that is associated with one or more sets of time-based data such as a plurality of predictive temporal parameters. For example, the input data object can include a grouping data structure that links one or more sets of time-based data, labels, and/or other characteristics that can be relevant to a predictive classification process. The input data object, for example, can include a grouping label for a plurality of predictive temporal parameters. In some examples, the input data object can include a unique identifier that is associated with one or more input data object parameters for facilitating the generation of a predictive classification through a predictive classification process. The input data object can be based on a machine-learning based model and/or a predictive classification process.

A predictive classification process, for example, can include a multi-stage predictive classification process configured to output a predictive classification for the input data object based on predictive temporal parameters associated with the input data object. As examples, the predictive classification process can include a medication adherence classification process, a medical disease classification process, and/or any other predictive classification process in which a classification can be derived from temporal observations. As one example, the predictive classification process can include a medication adherence classification process and the input data object can include a patient that is associated with physiological signals over a time period that can be predictive of whether the patient is taking a medication during and/or before the time period. For instance, the predictive classification process can be configured to generate a predictive classification for the patient that identifies whether the patient has completed a prescribed regimen based on predictive temporal parameters such as the physiological signals associated with the patient. In this example use case, the input data object can be representative of an individual that has generated the predictive temporal parameters through a CGM.

The term “input data object parameters” can refer to a data entity that describes a component associated with an input data object. The input data object parameters can describe a plurality of attributes for an input data object. For example, the input data object can include a grouping data structure that links one or more sets of time-based data, labels, and/or other characteristics that can be relevant to a predictive classification process. The input data object parameters can refer to the sets of time-based data, labels, and/or other characteristics that can be relevant to the predictive classification process.

In a medication adherence use case, the input data object can be representative of a patient and the input data object parameters can include a unique identifier, demographic information, electronic health records, and/or any other characteristic associated with the patient. In addition, or alternatively, the input data object parameters can include predictive temporal parameters for the input data object and/or one or more historical data objects or classification parameters for the input data object.

The term “predictive temporal parameters” can refer to a data entity that describes a predictive temporal component associated with an input data object that is predictive of a predictive classification. The predictive temporal parameters can include a set of time-based data that is associated with one or more particular times. For example, in some embodiments, the predictive temporal parameters can include a plurality of sensor measurements over an evaluation time period. The plurality of sensor measurements over the evaluation time period can be associated with a plurality of timestamps with respect to the evaluation time period. As one example, the sensor measurements can include CGM measurements. In some embodiments, for example, the predictive temporal parameters can include a CGM measurement every 5 minutes over a twenty-four hour time period. By way of example, the predictive temporal parameters can include timeseries data representative of a glucose concentration in an individual's interstitial fluid at one or more times throughout the twenty-four hour time period. The individual, for example, can be represented by an input data object with a unique identifier (“UID”).

The term “predictive classification parameters” can refer to a data entity that describes a classification of interest for the input data object. The predictive classification parameter can be based on a predictive classification process. As examples, the predictive classification parameters can be indicative of a medication that is being monitored for a medication adherence classification process, one or more diseases for a medical disease classification process, and/or the like. In a medical adherence use case, the predictive classification parameters can be indicative of the medications being taken by (or prescribed to) an individual with a UID. The individual's UID, for example, can correspond to an input data object that groups together the predictive temporal parameters and the predictive classification parameters for an individual that generated the predictive temporal parameters (e.g., using a CGM).

The term “machine-learning based temporal classification model” can refer to a data entity that describes parameters, hyper-parameters, and/or defined operations of a machine-learning based model (e.g., model including at least one of one or more rule-based layers, one or more layers that depend on trained parameters, etc.). The machine-learning based temporal classification model can be trained to perform an end-to-end predictive classification process configured to output a predictive classification for an input data object based on predictive temporal parameters. The machine-learning based temporal classification model can leverage predictive temporal parameters to generate a classification of interest based on the predictive classification process.

The machine-learning based temporal classification model can include one or more of any type of machine-learning based models including one or more supervised, unsupervised, semi-supervised, and/or reinforcement learning models. In some embodiments, the machine-learning based temporal classification model can include multiple models configured to perform one or more different stages of a multi-stage predictive classification process. By way of example, the machine-learning based temporal classification model can include (i) an input data object augmentation model configured to augment an input data object with additional input data parameters and/or (ii) a machine-learning based input data object classification model configured to generate a predictive classification of the input data object.

The term “input data object augmentation model” can refer to a data entity that describes parameters, hyper-parameters, and/or defined operations of a data augmentation model (e.g., model including at least one of one or more rule-based layers, one or more layers that depend on trained parameters, etc.). The input data object augmentation model can be configured to process an input data object and/or one or more input data object parameters to generate one or more augmented input data object parameters that are tailored for a predictive classification process. The augmented input data object parameters, for example, can include one or more augmented temporal data objects generated based on a plurality of predictive temporal parameters, one or more predictive data representations generated based on the augmented temporal data objects, and/or the like.

The term “augmented temporal data object” can refer to a data entity that describes one or more variations of the predictive temporal parameters associated with the input data object. By way of example, an augmented temporal data object can include a derivative, an integral, and/or one or more other transformations of the predictive temporal parameters using one or more transformation functions. Each transformation can emphasize one or more different features of the predictive temporal parameters. As examples, an augmented temporal data object can include one or more derivatives of the plurality of predictive temporal parameters. A first derivative can emphasize a rate of change (e.g., an acceleration) at one or more points within the plurality of predictive temporal parameters. A second derivative can emphasize a rate at which the plurality of predictive temporal parameters normalize and/or destabilize (e.g., based on one or more peaks/valleys, etc.). Other example transformations can emphasize spectral characteristics associated with the plurality of predictive temporal parameters, a response time (e.g., based on an area of a curve, etc.) associated with the plurality of predictive temporal parameters, and/or the like.

The term “predictive data representation” can refer to a data entity that describes a computer-interpretable representation for temporal data. For instance, a predictive data representation can include a multi-dimensional tensor. A predictive data representation, for example, can be indicative of a heat map for an augmented temporal data object and/or a plurality of predictive temporal parameters. In some embodiments, for example, the predictive data representation can include a heat map representative of a three-dimensional matrix in which a first dimension is represented by an x-axis of the heat map, a second dimension is represented by a y-axis of the heat map, and a third dimension is represented by a color intensity of the heat map. The predictive data representation can include a visual representation and/or any other computer-interpretable tensor representation. A heat map, for example, can be visualized by a computer in a plurality of different formats. In some embodiments, the data format of the heat map can be represented as a predictive image. For example, the predictive data representation can include a predictive image including a plurality of image pixels.

The heat map can be a three-dimensional tensor that includes (i) a first dimension indicative of a time associated with a predictive temporal parameter, (ii) a second dimension indicative of a scale of the predictive temporal parameter, and/or (iii) a third dimension indicative of a degree to which the scale of the predictive temporal parameter is represented by the plurality of predictive temporal parameters. For example, the heat map can be represented by a predictive image in which the horizontal (x) axis represents time, a vertical (y) axis represents scale, and/or a color intensity of a respective image pixel represents the degree to which the scale of the predictive temporal parameter is represented by the plurality of predictive temporal parameters.

Any combination of time and/or scale can be associated with multiple values, each of which can represent a degree to which a respective scale value is represented in the plurality of predictive temporal parameters. A respective scale value can represent a degree to which a wavelet (and/or another time-frequency transform) function is stretched or compressed. The degree to which the respective scale value is represented in the plurality of predictive temporal parameters can be determined using one or more wavelet transforms and/or other techniques for quantifying scale/frequency components for a datapoint. By way of example, the third dimension can be indicative of a wavelet transform coefficient of the first dimension and the second dimension of the predictive data representation. Wavelet transforms, for example, can include one technique for determining scale in the plurality of predictive temporal parameters. For example, wavelet transforms can utilize one or more different underlying shape functions to determine a scale present in a predictive temporal parameter at a given time.

The term “multi-channel predictive data representation” can refer to a data entity that describes a multi-channel data structure with a plurality of temporal features. The multi-channel predictive data representation, for example, can include a plurality of predictive data representations. For example, in the event that there are more than one set of predictive temporal parameters and/or augmented temporal data objects associated with an input data object, one or more of the predictive temporal parameters and/or augmented temporal data object can be represented as a separate predictive data representation (e.g., a heat map, etc.) to form an overlapping stack of predictive data representations (e.g., stacked heat maps). Each respective predictive data representation of the multi-channel predictive data representation can include a layer and/or channel of the multi-channel predictive data representation.

As one example, a multi-channel predictive data representation can include (i) a first channel (and/or layer) that includes a first predictive data representation generated from the plurality of predictive temporal parameters (e.g., with no derivative or integral taken), (ii) a second channel (and/or layer) that includes a second predictive data representation generated from a first augmented temporal data object (e.g., a first derivative of the plurality of predictive temporal parameters), (iii) a third channel (and/or layer) that includes a third predictive channel representation generated from a second augmented temporal data object (e.g., a second derivative of the plurality of predictive temporal parameters), and/or (iv) a fourth channel (and/or layer) that includes a fourth predictive channel representation generated from a third augmented temporal data object (e.g., a first integral of the plurality of predictive temporal parameters).

The term “machine-learning based input data object classification model” can refer to a data entity that describes parameters, hyper-parameters, and/or defined operations of a machine-learning based model (e.g., model including at least one of one or more rule-based layers, one or more layers that depend on trained parameters, etc.). The machine-learning based input data object classification model can include a portion of the machine-learning based temporal classification model. The machine-learning based input data object classification model can be trained to output a predictive classification for an input data object based on a multi-channel predictive data representation. For example, the machine-learning based input data object classification model can be configured to generate the predictive classification for the input data object based on the multi-channel predictive data representation and a predictive classification parameter that describes an intended predictive classification type.

The machine-learning based input data object classification model can include any type of machine-learning based model including one or more supervised, unsupervised, semi-supervised, and/or reinforcement learning models. In some implementations, the machine-learning based input data object classification model can include an image classification model such as one or more logistic regression models, naïve bayes models, K-nearest Neighbors, support vector machines, neural networks, classification models, and/or the like. By way of example, the image classification model can include a neural network such as a convolutional neural network (e.g., ResNets, etc.). The machine-learning based input data object classification model can be trained using one or more machine-learning based techniques. For example, the machine-learning based input data object classification model can be trained using supervised training techniques based on a historical data object including data indicative of a plurality of historical predictive classifications and a plurality of historical predictive temporal parameters.

The term “predictive classification” can refer to a data entity that describes an output of the machine-learning based temporal classification model. The predictive classification can include an attribute for the input data object that is derived from the predictive temporal parameters and can include any type of insight for an input data object that can be derived from temporal observations. The predictive classification can be based on the predictive classification parameter. As examples, the predictive classification can indicate whether a monitored medication is being taken by a patient, whether a patient has a particular disease, and/or the like. As one example, in a medication adherence use case, the predictive classification can be indicative of whether an individual is taking a medication such as insulin. By way of example, the predictive classification can predict whether a particular individual has taken a medication (e.g., insulin) before and/or during an evaluation time period (e.g., a 24-hour time period).

The term “historical data object” can refer to a data entity that describes historical training information for training and/or evaluating machine-learning based models such as those described herein. For example, the historical data object can include historical data associated with an intended operation of the machine-learning based temporal classification model. By way of example, the historical data object can include data indicative of a plurality of historical predictive classifications and/or a plurality of historical predictive temporal parameters. The historical data object, for example, can include a plurality of training pairs. Each training pair can include a historical predictive classification and a historical multi-channel predictive data representation corresponding to a respective plurality of historical predictive temporal parameters. As one example, in a medication adherence use case, a training pair can be indicative of: (i) a historical multi-channel predictive data representation and (ii) whether an individual corresponding to the historical multi-channel predictive data representation ingested a monitored medication (e.g., insulin) during an evaluation time period (e.g., 24-hour time period).

### II. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as in a particular directory, folder, or library. Software components may be static (e.g., pre-established or fixed) or dynamic (e.g., created or modified at the time of execution).

A computer program product may include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SWIM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### III. Example System Framework

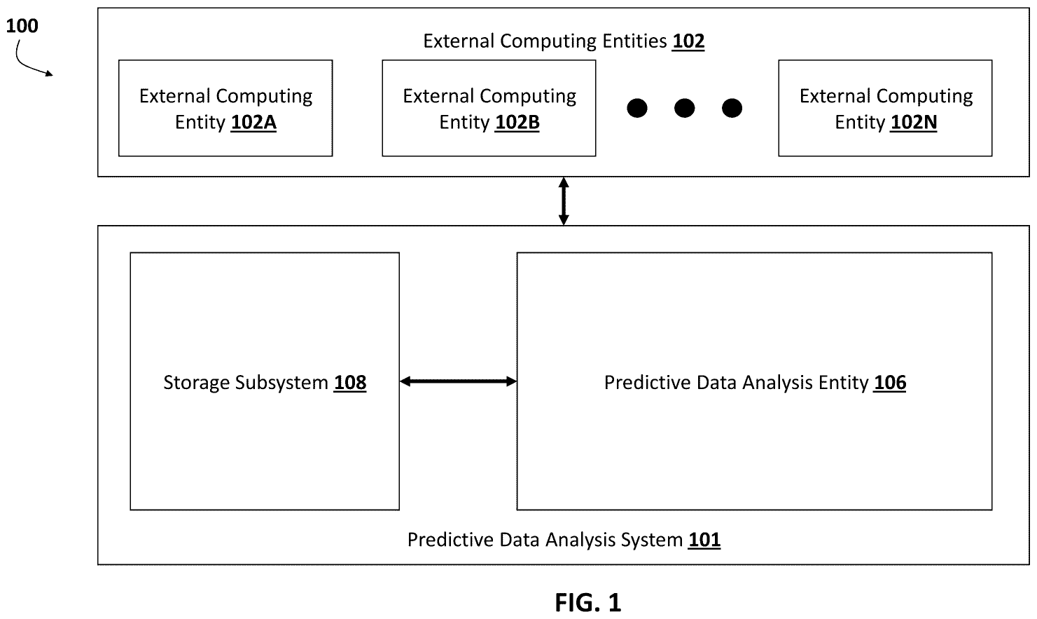

FIG. 1 provides an example overview of a system 100 that can be used to practice embodiments of the present disclosure. The system 100 includes a predictive data analysis system 101 comprising a predictive data analysis computing entity 106 configured to generate outputs that can be used to perform one or more output-based actions. The predictive data analysis system 101 may communicate with one or more external computing entities 102A-N using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (e.g., network routers, and/or the like).

The system 100 includes a storage subsystem 108 configured to store at least a portion of the data utilized by the predictive data analysis system 101. The predictive data analysis computing entity 106 may be in communication with the external computing entities 102A-N. The predictive data analysis computing entity 106 may be configured to: (i) train one or more machine learning models based on a training data store stored in the storage subsystem 108, (ii) store trained machine learning models as part of a model definition data store of the storage subsystem 108, (iii) utilize trained machine learning models to perform an action, and/or the like.

In one example, the system predictive data analysis computing entity 106 may be configured to generate a prediction, classification, and/or any other data insight based on data provided by an external computing entity such as external computing entity 102A, external computing entity 102B, and/or the like.

The storage subsystem 108 may be configured to store the model definition data store and the training data store for one or more machine learning models. The predictive data analysis computing entity 106 may be configured to receive requests and/or data from at least one of the external computing entities 102A-N, process the requests and/or data to generate outputs (e.g., predictive outputs, classification outputs, and/or the like), and provide the outputs to at least one of the external computing entities 102A-N. In some embodiments, the external computing entity 102A, for example, may periodically update/provide raw and/or processed input data to the predictive data analysis system 101. The external computing entities 102A-N may further generate user interface data (e.g., one or more data objects) corresponding to the outputs and may provide (e.g., transmit, send, and/or the like) the user interface data corresponding with the outputs for presentation to the external computing entity 102A (e.g., to an end-user).

The storage subsystem 108 may be configured to store at least a portion of the data utilized by the predictive data analysis computing entity 106 to perform one or more steps/operations and/or tasks described herein. The storage subsystem 108 may be configured to store at least a portion of operational data and/or operational configuration data including operational instructions and parameters utilized by the predictive data analysis computing entity 106 to perform the one or more steps/operations described herein. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

The predictive data analysis computing entity 106 can include an analysis engine and/or a training engine. The predictive analysis engine may be configured to perform one or more data analysis techniques. The training engine may be configured to train the predictive analysis engine in accordance with the training data store stored in the storage subsystem 108.

### Example Predictive Data Analysis Computing Entity

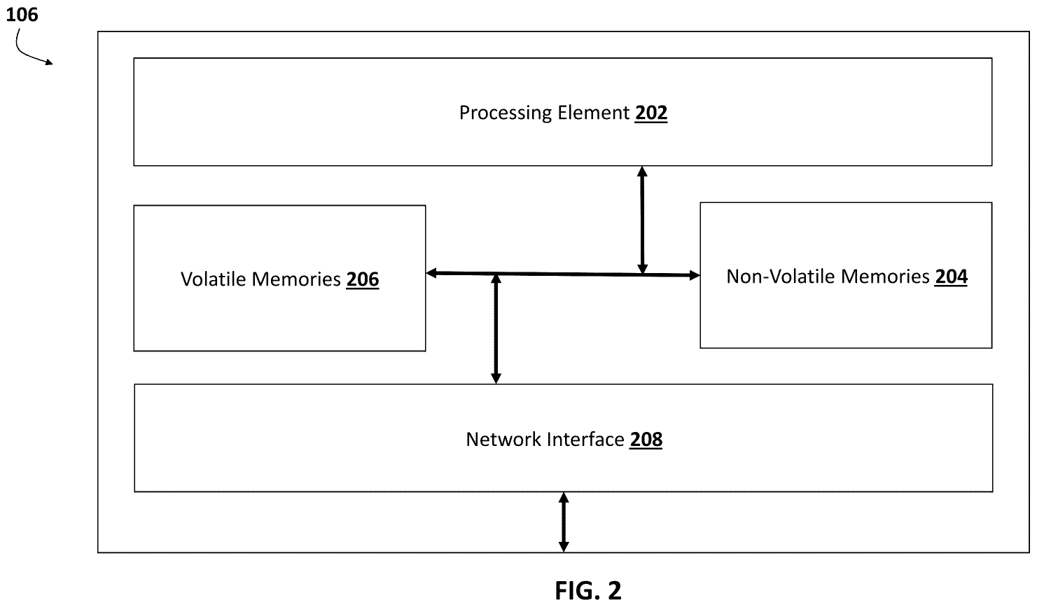

FIG. 2 provides an example predictive data analysis computing entity 106 in accordance with some embodiments discussed herein. In general, the terms computing entity, computer, entity, device, system, and/or similar words used herein interchangeably may refer to, for example, one or more computers, computing entities, desktops, mobile phones, tablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. Such functions, steps/operations, and/or processes may include, for example, transmitting, receiving, operating on, processing, displaying, storing, determining, creating/generating, monitoring, evaluating, comparing, and/or similar terms used herein interchangeably. In one embodiment, these functions, steps/operations, and/or processes can be performed on data, content, information, and/or similar terms used herein interchangeably.

The predictive data analysis computing entity 106 may include a network interface 208 for communicating with various computing entities, such as by communicating data, content, information, and/or similar terms used herein interchangeably that can be transmitted, received, operated on, processed, displayed, stored, and/or the like.

In one embodiment, the predictive data analysis computing entity 106 may include or be in communication with a processing element 202 (also referred to as processors, processing circuitry, and/or similar terms used herein interchangeably) that communicate with other elements within the predictive data analysis computing entity 106 via a bus, for example. As will be understood, the processing element 202 may be embodied in a number of different ways including, for example, as at least one processor/processing apparatus, one or more processors/processing apparatuses, and/or the like.

For example, the processing element 202 may be embodied as one or more complex programmable logic devices (CPLDs), microprocessors, multi-core processors, coprocessing entities, application-specific instruction-set processors (ASIPs), microcontrollers, and/or controllers. Further, the processing element 202 may be embodied as one or more other processing devices or circuitry. The term circuitry may refer to an entirely hardware embodiment or a combination of hardware and computer program products. Thus, the processing element 202 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 202 may be configured for a particular use or configured to execute instructions stored in one or more memory elements including, for example, one or more volatile memories 206 and/or non-volatile memories 204202. As such, whether configured by hardware or computer program products, or by a combination thereof, the processing element 202 may be capable of performing steps or operations according to embodiments of the present disclosure when configured accordingly. The processing element 202, for example in combination with the one or more volatile memories 206 and/or or non-volatile memories 204, may be capable of implementing one or more computer-implemented methods described herein. In some implementations, the predictive data analysis computing entity 106 can include a computing apparatus, the processing element 202 can include at least one processor of the computing apparatus, and the one or more volatile memories 206 and/or non-volatile memories 204 can include at least one memory including program code. The at least one memory and the program code can be configured to, upon execution by the at least one processor, cause the computing apparatus to perform one or more steps/operations described herein.

The non-volatile memories 204 (also referred to as non-volatile storage, memory, memory storage, memory circuitry, media, and/or similar terms used herein interchangeably) may include at least one non-volatile memory device 204, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile memories 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

The one or more volatile memories (also referred to as volatile storage, memory, memory storage, memory circuitry, media, and/or similar terms used herein interchangeably) can include at least one volatile memory 206 device, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile memories 206 may be used to store at least portions of the databases, database instances, database management systems, data, applications, programs, program modules, scripts, source code, object code, byte code, compiled code, interpreted code, machine code, executable instructions, and/or the like being executed by, for example, the processing element 202. Thus, the databases, database instances, database management systems, data, applications, programs, program modules, scripts, source code, object code, byte code, compiled code, interpreted code, machine code, executable instructions, and/or the like may be used to control certain embodiments of the operation of the predictive data analysis computing entity 106 with the assistance of the processing element 202.

As indicated, in one embodiment, the predictive data analysis computing entity 106 may also include the network interface 208 for communicating with various computing entities, such as by communicating data, content, information, and/or the like that can be transmitted, received, operated on, processed, displayed, stored, and/or the like. Such communication data may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

### Example External Computing Entity

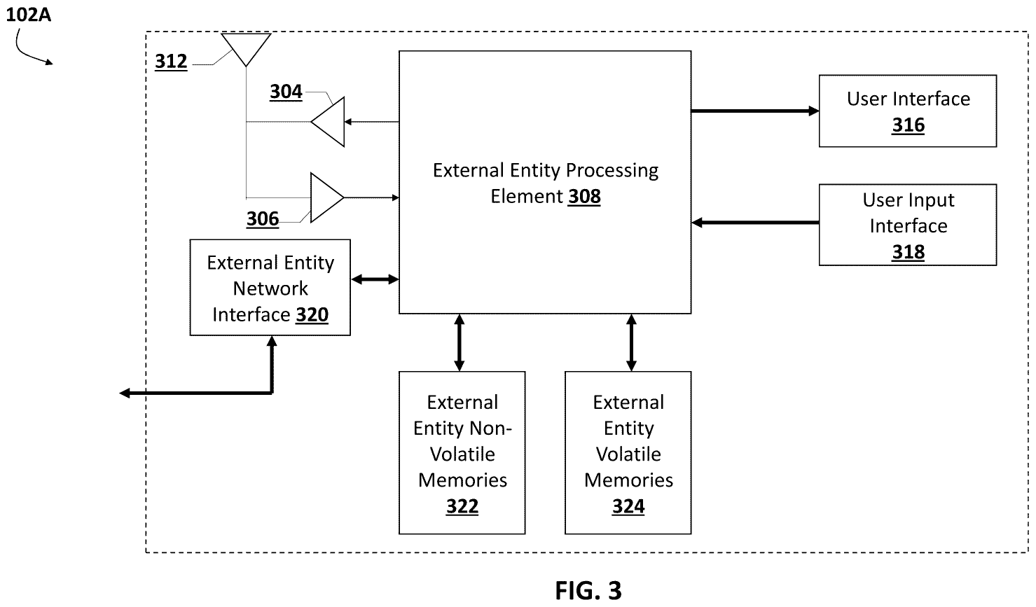

FIG. 3 provides an example external computing entity 102A in accordance with some embodiments discussed herein. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. The external computing entities 102A-N can be operated by various parties. As shown in FIG. 3, the external computing entity 102A can include an antenna 312, a transmitter 304 (e.g., radio), a receiver 306 (e.g., radio), and/or an external entity processing element 308 (e.g., CPLDs, microprocessors, multi-core processors, coprocessing entities, ASIPs, microcontrollers, and/or controllers) that provides signals to and receives signals from the transmitter 304 and the receiver 306, correspondingly. As will be understood, the external entity processing element 308 may be embodied in a number of different ways including, for example, as at least one processor/processing apparatus, one or more processors/processing apparatuses, and/or the like as described herein with reference the processing element 202.

The signals provided to and received from the transmitter 304 and the receiver 306, correspondingly, may include signaling information/data in accordance with air interface standards of applicable wireless systems. In this regard, the external computing entity 102A may be capable of operating with one or more air interface standards, communication protocols, modulation types, and access types. More particularly, the external computing entity 102A may operate in accordance with any of a number of wireless communication standards and protocols, such as those described above with regard to the predictive data analysis computing entity 106. In a particular embodiment, the external computing entity 102A may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102A may operate in accordance with multiple wired communication standards and protocols, such as those described above with regard to the predictive data analysis computing entity 106 via an external entity network interface 320.

Via these communication standards and protocols, the external computing entity 102A can communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102A can also download changes, add-ons, and updates, for instance, to its firmware, software (e.g., including executable instructions, applications, program modules), operating system, and/or the like.

According to one embodiment, the external computing entity 102A may include location determining embodiments, devices, modules, functionalities, and/or the like. For example, the external computing entity 102A may include outdoor positioning embodiments, such as a location module adapted to acquire, for example, latitude, longitude, altitude, geocode, course, direction, heading, speed, universal time (UTC), date, and/or various other information/data. In one embodiment, the location module can acquire data such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating a position of the external computing entity 102A in connection with a variety of other systems, including cellular towers, Wi-Fi access points, and/or the like. Similarly, the external computing entity 102A may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments can be used in a variety of settings to determine the location of someone or something to within inches or centimeters.

The external computing entity 102A may include a user interface 316 (e.g., a display, speaker, and/or the like) that can be coupled to the external entity processing element 308. In addition, or alternatively, the external computing entity 102A can include a user input interface 319 (e.g., keypad, touch screen, microphone, and/or the like) coupled to the external entity processing element 308).

For example, the user interface 316 may be a user application, browser, and/or similar words used herein interchangeably executing on and/or accessible via the external computing entity 102A to interact with and/or cause the display, announcement, and/or the like of information/data to a user. The user input interface 318 can comprise any of a number of input devices or interfaces allowing the external computing entity 102A to receive data including, as examples, a keypad (hard or soft), a touch display, voice/speech interfaces, motion interfaces, and/or any other input device. In embodiments including a keypad, the keypad can include (or cause display of) the conventional numeric (0-9) and related keys (#, *, and/or the like), and other keys used for operating the external computing entity 102A and may include a full set of alphabetic keys or set of keys that may be activated to provide a full set of alphanumeric keys. In addition to providing input, the user input interface 318 can be used, for example, to activate or deactivate certain functions, such as screen savers, sleep modes, and/or the like.

The external computing entity 102A can also include one or more external entity non-volatile memories 322 and/or one or more external entity volatile memories 324, which can be embedded within and/or may be removable from the external computing entity 102A. As will be understood, the external entity non-volatile memories 322 and/or the external entity volatile memories 324 may be embodied in a number of different ways including, for example, as described herein with reference the non-volatile memories 204 and/or the external volatile memories 206.

### IV. Example System Operations

As described below, various embodiments of the present disclosure leverage machine-learning based techniques to make important technical contributions to data and data processing intensive investigative processes.

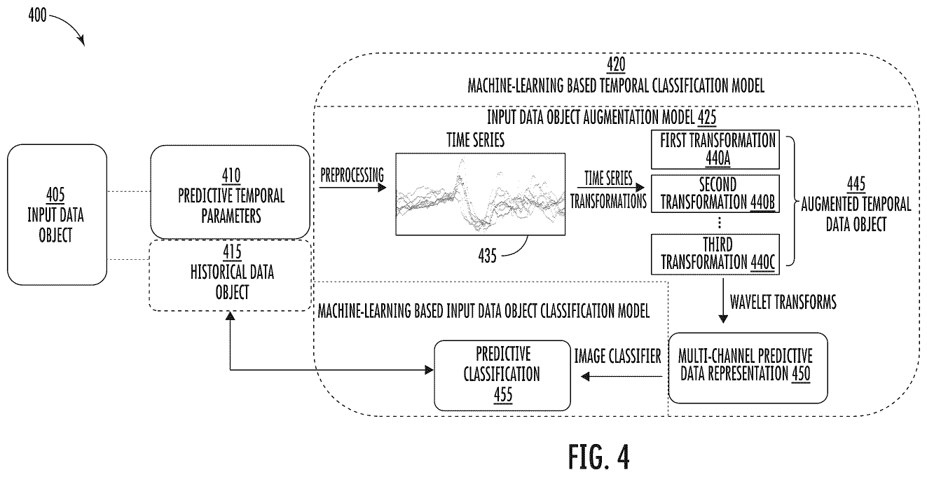

FIG. 4 provides a dataflow diagram 400 of an example predictive classification process for generating a predictive classification based on time-based data in accordance with some embodiments discussed herein. The dataflow diagram 400 depicts an end-to-end predictive classification process for building useful temporal features from time-based data and generating predictions for the time-based data based on the temporal features. The predictive classification process includes one or more stages. In a first stage, a multi-channel predictive data representation 450 including useful temporal features can be generated for an input data object 405. In a second stage, a machine-learning based input data object classification model 430 can be trained to output a predictive classification 455 based on the multi-channel predictive data representation 450. Once trained, the machine-learning based input data object classification model 430 can be used to generate the predictive classification 455 for unlabeled multi-channel predictive data representations to make inferences for the input data object 405 based on a plurality of predictive temporal parameters 410.

Each stage of the predictive classification process can be performed by at least a portion of a machine-learning based temporal classification model 420. The machine-learning based temporal classification model 420 can be implemented by one or more computing devices and/or systems described herein. For example, the predictive data analysis computing entity 106 can utilize the machine-learning based temporal classification model 420 to overcome the various limitations with conventional prediction and data processing techniques.

The machine-learning based temporal classification model 420 can receive the input data object 405 that is associated with a predictive classification process. The input data object 405 can include a data entity that describes an initial input to the machine-learning based temporal classification model 420 that is associated with a time-based classification process.

The input data object 405 can include a data entity that describes an initial input to the machine-learning based temporal classification model 420. The input data object 405 can represent a data entity that is associated with one or more sets of time-based data such as the predictive temporal parameters 410. For example, the input data object 405 can include a grouping data structure that links one or more sets of time-based data, labels, and/or other characteristics that can be relevant to a predictive classification process. The input data object 405, for example, can include a grouping label (e.g., a UID) for the predictive temporal parameters 410. In some examples, the input data object 405 can include a unique identifier that is associated with one or more input data object parameters for facilitating the generation of the predictive classification 455 through a predictive classification process. The input data object 405 can be based on a machine-learning based model and/or a predictive classification process.

A predictive classification process, for example, can include a multi-stage predictive classification process configured to output a predictive classification for the input data object 405 based on the predictive temporal parameters 410 associated with the input data object 405. As examples, the predictive classification process can include a medication adherence classification process, a medical disease classification process, and/or any other predictive classification process in which a predictive classification can be derived from temporal observations. As one example, the predictive classification process can include a medication adherence classification process and the input data object 405 can include a patient that is associated with physiological signals over a time period that can be predictive of whether the patient is taking a medication during and/or before the time period. For instance, the machine-learning based temporal classification model 420 can be configured to generate a predictive classification for a patient that identifies whether the patient has completed a prescribed regimen based on the predictive temporal parameters 410 such as the physiological signals associated with the patient. In this example use case, the input data object 405 can be representative of an individual that has generated the predictive temporal parameters 410 through a CGM.

The input data object 405 can be associated with the predictive temporal parameters 410 that describe a predictive temporal component that is predictive of the predictive classification 455. The plurality of predictive temporal parameters 410 can include a set of time-based data that is associated with one or more particular times. For example, in some embodiments, the predictive temporal parameters 410 can include a plurality of sensor measurements over an evaluation time period. The plurality of sensor measurements over the evaluation time period can be associated with a plurality of timestamps with respect to the evaluation time period. As one example, the sensor measurements can include CGM measurements. In some embodiments, for example, the predictive temporal parameters 410 can include a CGM measurement every 5 minutes over a twenty-four hour time period. By way of example, the predictive temporal parameters can include timeseries data representative of a glucose concentration in an individual's interstitial fluid at one or more times throughout the twenty-four hour time period. The individual, for example, can be represented by the input data object 405 with a UID.

The input data object 405 can be associated with predictive classification parameters that can describe a classification of interest for the input data object 405. The predictive classification parameters can be based on a predictive classification process. As examples, the predictive classification parameters can be indicative of a medication that is being monitored for a medication adherence classification process, one or more diseases for a medical disease classification process, and/or the like. In a medical adherence use case, the predictive classification parameters can be indicative of the medications being taken by (or prescribed to) an individual with a UID. The individual's UID, for example, can correspond to the input data object 405 that groups together the predictive temporal parameters 410 and the predictive classification parameters for an individual that generated the predictive temporal parameters 410 (e.g., using a CGM).

A historical data object 415 can describe historical training information for training and evaluating one or more portions of the machine-learning based temporal classification model 420. The historical data object 415, for example, can include one or more training labels for the input data object 405 and/or the predictive temporal parameters 410. The training labels can include a known predictive classification. By way of example, the training label can indicate whether a patient is actually adhering to a medical regime in a medication adherence classification process, etc. In some embodiments, the input data object 405, for example in a training/evaluation phase mode, can be associated with the historical data object 415 (e.g., labeled predictive temporal parameters). In addition, or alternatively, the input data object 405, for example in a classification phase, cannot have access to the historical data object 415 (e.g., unlabeled predictive temporal parameters).

The machine-learning based temporal classification model 420 can determine one or more augmented temporal data objects 445 based on the predictive temporal parameters 410. In some embodiments, at least a portion of the machine-learning based temporal classification model 420 can be configured to determine the augmented temporal data objects 445. For example, the machine-learning based temporal classification model 420 can include one or more different models which can be configured to collectively perform the end-to-end predictive classification process. By way of example, the machine-learning based temporal classification model 420 can include (i) an input data object augmentation model 425 configured to augment the input data object 405 with additional input data parameters such as temporal features useful for the predictive classification process and/or (ii) the machine-learning based input data object classification model 430 configured to generate the predictive classification 455 of the input data object 405.

The input data object augmentation model 425 can include a data entity that describes parameters, hyper-parameters, and/or defined operations of a data augmentation model. The input data object augmentation model 425 can be configured to process the input data object 405 and/or one or more input data object parameters of the input data object 405 to generate one or more augmented input data object parameters that are tailored for the predictive classification process. The augmented input data object parameters, for example, can include the augmented temporal data objects 445 generated based on the predictive temporal parameters 410, one or more predictive data representations generated based on the augmented temporal data objects 445, the multi-channel predictive data representation 450, and/or the like.

In some embodiments, the machine-learning based temporal classification model 420 (e.g., the input data object augmentation model 425) can preprocess the predictive temporal parameters 410 to generate a time-series model 435. For example, as described herein, the predictive temporal parameters 410 can include a plurality of sensor measurements over an evaluation time period that are associated with a plurality of timestamps with respect to the evaluation time period. The machine-learning based temporal classification model 420 (e.g., the input data object augmentation model 425) can generate the time-series model 435 for the sensor measurements based on the timestamps with respect to the evaluation time period. The machine-learning based temporal classification model 420 (e.g., the input data object augmentation model 425) can determine the augmented temporal data objects 445 based on the time-series model 435.

The plurality of predictive temporal parameters 410 can be preprocessed to improve the accuracy (including precision, recall, and/or other measures) of the predictive classification 455. To do so, the predictive temporal parameters 410 can be aligned in one or more sub-alignments based on one or more patterns associated with the predictive temporal parameters 410. By way of example, the predictive temporal parameters 410 can include physiologic signals such as CGM signals that can be impacted by one or more activities throughout the evaluation time period. For instance, CGM data can be characterized by a glucose concentration peak following a time period of lower glucose levels and/or lower variability. Behaviorally, this can correspond to a common practice of eating breakfast after waking from a night's sleep. By aligning the predictive temporal parameters 410 in multiple segments according to a first pronounced peak of an evaluation time period, the machine-learning based temporal classification model 420 can preserve meaningful physiological characteristics of the predictive temporal parameters 410 in the aggregate and improve the accuracy of resulting classifications.

In some embodiments, the machine-learning based temporal classification model 420 (e.g., the input data object augmentation model 425) can select one or more different portions of the predictive temporal parameters 410. By way of example, the input data object augmentation model 425 can include one or more machine-learning based selection models configured to identify and/or select one or more optimal classification portions of the predictive temporal parameters 410 for the predictive classification process. For example, variability of glucose concentrations during sleep can be lower than waking variability for a patient. In such a case, the input data object augmentation model 425 can identify and/or select one or more portions of the predictive temporal parameters 410 that are generated during sleep to reduce uninformative variability in the predictive temporal parameters 410.

In some embodiments, the optimal classification portions of the predictive temporal parameters 410 can be selected based on contextual data associated with the predictive temporal parameters 410. The contextual data, for example, can be indicative of one or more associated activities, geographic locations, and/or other attributes associated with at least a portion of the predictive temporal parameters 410. By way of example, the contextual data can include acceleration data from a wearable activity tracker that can be used to infer when the individual is sleeping to enable the verified selection of the optimal classification portions of the predictive temporal parameters 410 that are generated during sleep.

The machine-learning based temporal classification model 420 (e.g., the input data object augmentation model 425) can generate the time-series model 435 based on the optimal classification portions of the predictive temporal parameters 410. The machine-learning based temporal classification model 420 (e.g., the input data object augmentation model 425) can determine the augmented temporal data objects 445 based on the time-series model 435 and/or the optimal classification portions of the predictive temporal parameters 410.

The augmented temporal data objects 445 can include at least one transformation (e.g., a derivative, integral, etc.) of the predictive temporal parameters 410. By way of example, the augmented temporal data objects 445 can include a first transformation 440A of the predictive temporal parameters 410, a second transformation 440B of the predictive temporal parameters 410, and/or a third transformation 440C of the predictive temporal parameters 410.

The augmented temporal data objects 445 can describe one or more variations of the predictive temporal parameters 410 (and/or optimal classification portions thereof) associated with the input data object 405. By way of example, a respective augmented temporal data object can include a derivative, an integral, and/or some other transformation of the predictive temporal parameters 410 using one or more transformation functions. Each transformation can emphasize one or more different features of the predictive temporal parameters. As examples, an augmented temporal data object can include one or more derivatives of the predictive temporal parameters 410. A first derivative (e.g., the first transformation 440A) can emphasize a rate of change (e.g., an acceleration) at one or more points within the predictive temporal parameters 410. A second derivative (e.g., the second transformation 440B) can emphasize a rate at which the predictive temporal parameters 410 normalize and/or destabilize (e.g., based on one or more peaks/valleys, etc.). Other derivatives (e.g., the third transformation 440C) can emphasize spectral characteristics associated with the predictive temporal parameters 410, a response time (e.g., based on an area of a curve, etc.) associated with the predictive temporal parameters 410, and/or the like.

The machine-learning based temporal classification model 420 (e.g., the input data object augmentation model 425) can generate one or more predictive data representations for the input data object based on the predictive temporal parameters 410 and/or the augmented temporal data objects 445. In addition, or alternatively, the machine-learning based temporal classification model 420 (e.g., the input data object augmentation model 425) can generate the multi-channel predictive data representation 450 based on the predictive data representations for the input data object 405.

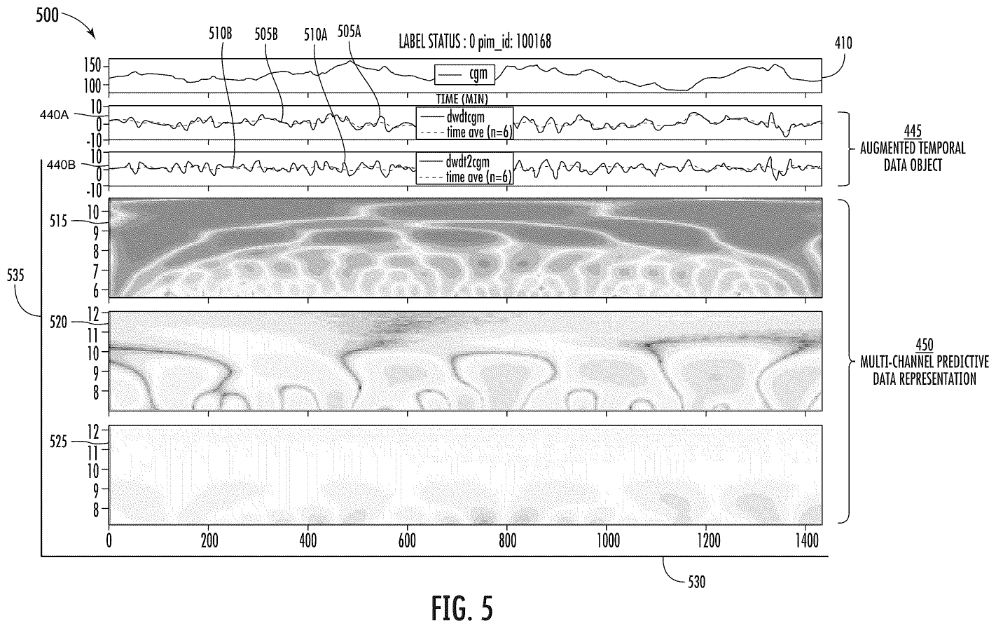

FIG. 5 provides an operational example 500 of a multi-channel predictive data representation in accordance with some embodiments discussed herein. The operational example 500 includes the predictive temporal parameters 410, the augmented temporal data objects 445, and the multi-channel predictive data representation 450. The multi-channel predictive data representation 450 includes a plurality of channels, each channel including a respective predictive data representation such as a first predictive data representation 515, a second predictive data representation 520, and/or a third predictive data representation 525.

A predictive data representation can describe a computer-interpretable representation for temporal data. For example, a respective predictive data representation can include a multi-dimensional tensor. The predictive data representation, for example, can be indicative of a heat map for the augmented temporal data objects 445 and/or the predictive temporal parameters 410. In some embodiments, for example, a predictive data representation can include a heat map representative of a three-dimensional matrix in which a first dimension is represented by an x-axis 530 of the heat map, a second dimension is represented by a y-axis 535 of the heat map, and a third dimension is represented by a color intensity of the heat map. The heat map can include a visual representation and/or any other computer-interpretable tensor representation. The heat map, for example, can be visualized by a computer in a plurality of different formats. In some embodiments, the data format of the heat map can be represented as an image. For example, the predictive data representation can include a predictive image including a plurality of image pixels.

The heat map can be a three-dimensional tensor that includes (i) a first dimension (e.g., the x-axis 530) indicative of a time associated with a predictive temporal parameter, (ii) a second dimension (e.g., the y-axis 535) indicative of a scale of the predictive temporal parameter, and/or (iii) a third dimension indicative of a degree to which the scale of the predictive temporal parameter is represented by the predictive temporal parameters 410. For example, the heat map can be represented by a predictive image in which the horizontal, the x-axis 530 represents time, a vertical, the y-axis 535 represents scale, and/or a color intensity represents the degree to which the scale of the predictive temporal parameter is represented by the predictive temporal parameters 410. By way of example, an x-axis coordinate of an image pixel can be indicative of the first dimension, a y-axis coordinate of the image pixel can be indicative of the second dimension, and a color intensity of the image pixel can be indicative of the third dimension.

Any combination of time and/or scale can be associated with multiple values, each of which can represent a degree to which a respective scale value is represented in the predictive temporal parameters 410. A respective scale value can represent a degree to which a wavelet (and/or another time-frequency transform) function is stretched or compressed. The degree to which the respective scale value is represented in the predictive temporal parameters 410 can be determined using one or more wavelet transforms and/or other techniques for quantifying scale/frequency components for a datapoint. By way of example, the third dimension can be indicative of a wavelet transform coefficient of the first dimension and the second dimension of the predictive data representation. Wavelet transforms, for example, can include one technique for determining scale in the predictive temporal parameters 410. The wavelet transforms can utilize one or more different underlying shape functions to determine a scale present in a predictive temporal parameter at a given time.

In the operational example 500, the machine-learning based temporal classification model 420 (e.g., the input data object augmentation model 425) can convert the predictive temporal parameters 410 into a first transformation 440A440A and a second transformation 440B440B. The first transformation 440A can include a first derivative of the predictive temporal parameters 410. For example, a first line 505A in the representation of the first transformation 440A can be representative of the first derivative of the predictive temporal parameters 410. A second line 505B in the representation of the first transformation 440A can be representative of a time-averaged version of the first derivative. The second transformation 440B can include a second derivative of the predictive temporal parameters 410. For example, a first line 510A in the representation of the second transformation 440B can be representative of the second derivative of the predictive temporal parameters 410. A second line 510B in the representation of the second transformation 440B can be representative of a time-averaged version of the second derivative.

The machine-learning based temporal classification model 420 can convert: (i) the predictive temporal parameters 410 into the first predictive data representation 515 (e.g., a first heat map, etc.), (ii) the first transformation 440A into the second predictive data representation 520 (e.g., a second heat map, etc.), and/or (iii) the second transformation 440B into the third predictive data representation 525 (e.g., a third heat map, etc.).

The first predictive data representation 515, for example, can be generated using a wavelet transform of the predictive temporal parameters 410. The second predictive data representation 520 can be generated from a wavelet transform of the time-averaged first derivative. The third predictive data representation 525 can be generated from a wavelet transform of the time-averaged second derivative. By way of example, the predictive data representations can be generated from the smoothed (time averaged) plots to reduce noise. In this manner, predictive data representations can emphasize important local properties (e.g., increase/decrease and/or concavity) without dramatic jumps.

In some embodiment, the machine-learning based temporal classification model 420 can compare wavelet transforms that use different underlying functions to identify an optimal type of transformation function that optimizes the predictive classification for the predictive temporal parameters 410, the first transformation 440A, and/or the second transformation 440B. By way of example, in some embodiments, the machine-learning based temporal classification model 420 can utilize one or more different wavelets for each temporal sequence of data (e.g., the predictive temporal parameters 410, the first transformation 440A, and/or the second transformation 440B). A type of wavelet transform used for each temporal sequence of data, for example, can be determined in an automated fashion to improve model accuracy. By way of example, the machine-learning based temporal classification model 420 can intelligently select a particular wavelet transform for a particular temporal sequence of data based on one or more attributes of the particular temporal sequence of data. The attributes, for example, can be indicative of a shape (e.g., number/magnitude of one or more peaks and/or valleys) of the particular temporal sequence of data, a variability of the particular temporal sequence of data, historical results associated with the particular temporal sequence of data, etc.

Each type of wavelet transform can be used for each temporal sequence of data over a uniform time period (e.g., the evaluation time period). In addition, or alternatively, different wavelet transforms can be utilized for different time windows of the evaluation time period. For example, a wavelet transform can include a short-time Fourier transform (FT) in which a temporal sequence of data can be broken into time windows. The FT can be applied to each time window to generate a respective predictive data representation using the magnitude of the FT for each frequency within each time window.

In example predictive data representations, a cooler color can indicate a higher representation of a given scale and warmer colors can indicate a lower representation of a given scale (e.g., purple>blue>green>yellow>orange>red). In alternative embodiments, any number of separate colors (e.g., red, green, blue) can be utilized to represent different temporal sequence of data (e.g., the predictive temporal parameters 410, the augmented temporal data objects 445) and the intensity of the color can represent the degree to which a given frequency is present within the temporal sequence of data.

The first predictive data representation 515, the second predictive data representation 520, and/or the third predictive data representation 525 can form the multi-channel predictive data representation 450 (e.g., a stack of heat maps) for the input data object. The multi-channel predictive data representation 450 describes a plurality of predictive data representations. For example, in the event that there are more than one set of predictive temporal parameters and/or augmented temporal data objects associated with an input data object, one or more of the predictive temporal parameters 410 and/or the augmented temporal data objects 445 can be represented as a separate predictive data representation (e.g., a heat map, etc.) to form an overlapping stack of predictive data representations (e.g., heat maps). Each respective predictive data representation of the multi-channel predictive data representation 450 can include a layer and/or channel of the multi-channel predictive data representation 450.

As one example, the multi-channel predictive data representation 450 can include (i) a first channel (and/or layer) that includes the first predictive data representation 515 generated from the predictive temporal parameters 410 (e.g., with no derivative or integral taken), (ii) a second channel (and/or layer) that includes the second predictive data representation 520 generated from a first augmented temporal data object (e.g., the first transformation 440A of the predictive temporal parameters 410), (iii) a third channel (and/or layer) that includes the third predictive data representation 525 generated from a second augmented temporal data object (e.g., the second transformation 440B of the predictive temporal parameters 410), and/or a fourth channel (and/or layer) that includes a fourth predictive data representation (not depicted) generated from a third augmented temporal data object (e.g., a first integral of the predictive temporal parameters 410).

The operational example 500 includes the multi-channel predictive data representation 450 that includes a plurality of predictive data representations derived from one type of sensor data such as CGM data. In addition, or alternatively, the multi-channel predictive data representation 450 can include layers to the stack that are generated from timeseries data of a different sensor type such as photoplethysmography (“PPG”) timeseries data, and/or the like. For example, in some embodiments, a predictive data representation can be generated from PPG timeseries data (or derivatives, integrals, and/or other transformations thereof) and included in the multi-channel predictive data representation 450.

Turning back to FIG. 4, the machine-learning based temporal classification model 420 (e.g., the machine-learning based input data object classification model 430) can generate the predictive classification 455 for the input data object 405 based on the multi-channel predictive data representation 450 and/or a predictive classification parameter. For example, the multi-channel predictive data representation 450 can be input to the machine-learning based input data object classification model 430 to generate the predictive classification 455 for the input data object 405.

The predictive classification 455 can describe an output of the machine-learning based temporal classification model 420. The predictive classification 455 can include an attribute for the input data object 405 that is derived from the predictive temporal parameters 410 and can include any type of insight for the input data object 405 that can be derived from temporal observations. The predictive classification 455 can be based on a predictive classification parameter. As examples, the predictive classification 455 can be whether a monitored medication is being taken by a patient, whether a patient has a particular disease, and/or the like.

The machine-learning based input data object classification model 430 can include a portion of the machine-learning based temporal classification model 420. The machine-learning based input data object classification model 430 can be trained to output the predictive classification 455 for the input data object 405 based on the multi-channel predictive data representation 450. For example, the machine-learning based input data object classification model 430 can be configured to generate the predictive classification 455 for the input data object 405 based on the multi-channel predictive data representation 450 and/or a predictive classification parameter.

The machine-learning based input data object classification model 430 can include any type of machine-learning based model including one or more supervised, unsupervised, semi-supervised, and/or reinforcement learning models. In some implementations, the machine-learning based input data object classification model 430 can include an image classification model such as one or more logistic regression models, naïve bayes models, K-nearest Neighbors, support vector machines, neural networks, classification models, and/or the like. By way of example, the image classification model can include a neural network such as a convolutional neural network (e.g., ResNets, etc.).

The machine-learning based input data object classification model 430 can be trained using one or more machine-learning based techniques. For example, the machine-learning based input data object classification model 430 can be trained using supervised training techniques based on the historical data object 415 including data indicative of a plurality of historical predictive classifications and a plurality of historical predictive temporal parameters. For example, the historical data object 415 can include historical data associated with an intended operation of the machine-learning based input data object classification model 430. By way of example, the historical data object 415 can include data indicative of a plurality of historical predictive classifications and/or a plurality of historical predictive temporal parameters. The historical data object 415, for example, can include a plurality of training pairs. Each training pair can include a historical predictive classification and a historical multi-channel predictive data representation corresponding to a respective plurality of historical predictive temporal parameters.

In some embodiments, for example, the historical data object 415 can include a ground truth predictive classification for the multi-channel predictive data representation 450. By way of example, the ground truth predictive classification can be indicative of whether a patient has been taking a prescribed medication. Using this knowledge, the machine-learning based input data object classification model 430 can be trained to classify the multi-channel predictive data representation 450 as to a probability that the input data object 405 associated with the multi-channel predictive data representation 450 has been taking the prescribed medication during and/or before the evaluation time period associated with the predictive temporal parameters 410. This can be performed for a plurality of labeled multi-channel predictive data representations (e.g., training pairs) to continually improve the accuracy of the machine-learning based input data object classification model 430. In some embodiments, during training, one or more channels of the multi-channel predictive data representation 450 can be weighted to improve the performance (e.g., accuracy, speed, etc.) of the machine-learning based input data object classification model 430.

The trained machine-learning based input data object classification model can be utilized to generate the predictive classification 455 for an unlabeled multi-channel predictive data representation. The predictive classification 455 can include an attribute for the input data object 405 that is derived from the predictive temporal parameters 410 and can include any type of insight for the input data object 405 that can be derived from temporal observations. The predictive classification 455 can be based on the predictive classification parameter. As examples, the predictive classification 455 can indicate whether a monitored medication is being taken by a patient, whether a patient has a particular disease, and/or the like. As one example, in a medication adherence use case, the predictive classification 455 can be indicative of whether an individual is taking a medication such as insulin. By way of example, the predictive classification 455 can predict whether a particular individual has taken a medication (e.g., insulin) before and/or during an evaluation time period (e.g., a 24-hour time period).

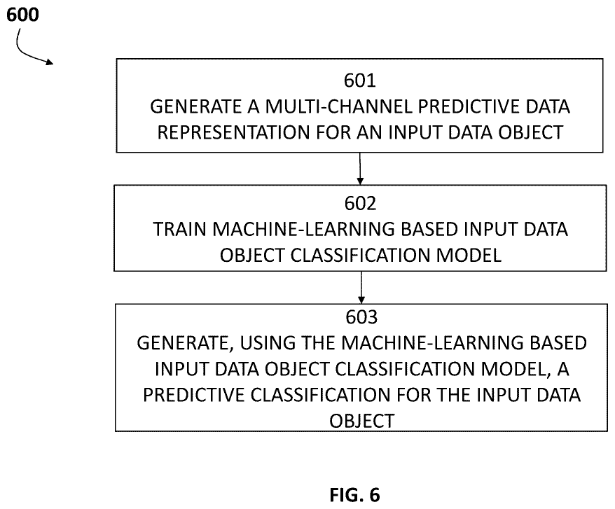

FIG. 6 is a flowchart showing an example of a process 600 for generating a predictive classification based on time-based data in accordance with some embodiments discussed herein. Via the various steps/operations of the process 600, the predictive data analysis computing entity 106 can implement a new data processing and prediction scheme to overcome the various limitations with conventional computer prediction techniques. By way of example, due to an inability to accurately process or interpret time-based data such as physiological signals for a predictive classification, conventional computer prediction techniques for generating predictive classifications can rely of secondary factors (e.g., pill counts, self-reporting, and/or the like) that may be weakly or inaccurately correlated to the predictive classification. Such techniques can be inaccurate, time-consuming, and unreliable and can be improved upon by the new data processing and prediction scheme described herein.

At step/operation 601, the process 600 includes generating a multi-channel predictive data representation for an input data object. For example, the predictive data analysis computing entity 106 can generate the multi-channel predictive data representation (e.g., the multi-channel predictive data representation 450) for the input data object as described herein.

At step/operation 602, the process 600 includes training a machine-learning based input data object classification model. For example, the predictive data analysis computing entity 106 can train the machine-learning based input data object classification model (e.g., the machine-learning based input data object classification model 430) using the multi-channel predictive data representation for the input data object as described herein.

As an example, the predictive data analysis computing entity 106 can train the machine-learning based input data object classification model using the multi-channel predictive data representation for the input data object. The machine-learning based input data object classification model can include a supervised image classification model. The predictive data analysis computing entity 106 can train the supervised image classification model to classify an input data object according to a predictive classification (e.g., a label of interest). Unlike traditional image classification models which are trained to generate a classification on the pixels in a single, two-dimensional image, the image classification model of the present disclosure is trained to generate the predictive classification for a plurality of predictive data representations grouped by the multi-channel predictive data representation.

At step/operation 603, the process 600 includes generating, using a machine-learning based input data object classification model, a predictive classification (e.g., the predictive classification 455) for the input data object based on the multi-channel predictive data representation. For example, once trained, the predictive data analysis computing entity 106 can generate, using the machine-learning based input data object classification model, the predictive classification for the input data object based on the multi-channel predictive data representation as described herein. For example, the predictive data analysis computing entity 106 can utilize a trained machine-learning based input data object classification model to generate a predictive classification from time-based data that has been transformed into a multi-channel predictive data representation (e.g., stacks of heat maps) as described herein.

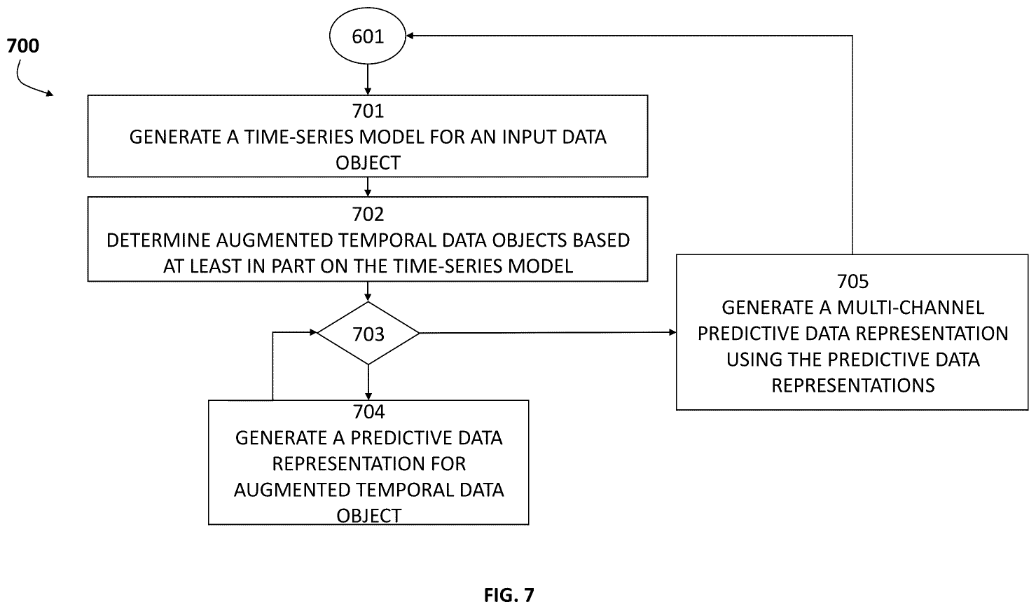

FIG. 7 is a flowchart showing an example of a process 700 for generating a multi-channel predictive data representation in accordance with some embodiments discussed herein. Via the various steps/operations of the process 700, the predictive data analysis computing entity 106 can implement at least a portion of a new data processing and prediction scheme to overcome the various limitations with conventional computer prediction techniques. In some embodiments, the process 700 can include a plurality of operations subsequent to the step/operation 601, where the process 600 includes generating a multi-channel predictive data representation for an input data object. In addition, or alternatively, the process 700 can include one or more suboperations of the step/operation 601.

At step/operation 701, the process 700 includes generating a time-series model for an input data object. For example, the predictive data analysis computing entity 106 can generate the time-series model (e.g., the time-series model 435) for the input data object as described herein. The predictive data analysis computing entity 106, for example, can receive an input data object associated with a predictive classification parameter and a plurality of predictive temporal parameters for the input data object. The plurality of predictive temporal parameters, for example, can include a plurality of sensor measurements over the evaluation time period that are associated with a plurality of timestamps with respect to the evaluation time period. The predictive data analysis computing entity 106 can generate a time-series model for the sensor measurements based on the timestamps with respect to the evaluation time period. The time-series model, for example, can include a two-dimensional time plot in which an x-axis represents a time of a sensor measurement and a y-axis represents a magnitude of the sensor measurement.

At step/operation 702, the process 700 includes determining augmented temporal data objects (e.g., the augmented temporal data objects 445) based on the time-series model. For example, the predictive data analysis computing entity 106 can determine the augmented temporal data objects based on the time-series model as described herein. The predictive data analysis computing entity 106, for example, can determine one or more augmented temporal data objects based on the predictive temporal parameters 410. The augmented temporal data objects 445 can include at least one derivative of the predictive temporal parameters 410.

At step/operations 703 and 704, the process 700 includes processing each augmented temporal data object to generate a predictive data representation (e.g., the multi-channel predictive data representation 450) for the respective augmented data object. For example, at 703, the predictive data analysis computing entity 106 can determine whether an augmented temporal data object does not have a corresponding predictive data representation. In response to identifying an augmented temporal data object that does not have a corresponding predictive data representation, at 704, the predictive data analysis computing entity 106 can generate a predictive data representation for the augmented temporal data object.

The predictive data analysis computing entity 106 can generate a predictive data representation for each augmented temporal data object as described herein. The predictive data analysis computing entity 106, for example, can generate one or more predictive data representations for the input data object based on the predictive temporal parameters 410 and the augmented temporal data objects 445. For example, the predictive data representation can include a heat map. In some embodiments, the first heat map can be generated from the predictive temporal parameters 410 (e.g., raw timeseries data with no derivative or integral taken), a second heat map can be generated from a first augmented data object (e.g., a first derivative of the raw timeseries data), a third heat map can be generated from a second augmented data object (e.g., a second derivative of the raw timeseries data), and a fourth heat map can be generated from third augmented data object (e.g., a first integral of the raw timeseries data).

At step/operation 705, the process 700 includes generating a multi-channel predictive data representation using the predictive data representations. For example, the predictive data analysis computing entity 106 can generate the multi-channel predictive data representation using the predictive data representations as described herein. The predictive data analysis computing entity 106, for example, can generate the multi-channel predictive data representation based on the predictive data representations for the input data object. In some embodiments, the multi-channel predictive data representation can include a plurality of different layers in a stack that may be weighted differently relative to one another. Differential weighting of each layer can improve the accuracy of models trained using the multi-channel predictive data representation. As described herein, weighting can be determined in an automated fashion to determine a weighting profile that maximizes model accuracy.

### V. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for machine-learning based input data augmentation and analysis, the computer-implemented method comprising:
determining, using one or more processors, one or more augmented temporal data objects based on a plurality of predictive temporal parameters of an input data object associated with a predictive classification process, wherein the augmented temporal data objects comprise at least one transformation of the predictive temporal parameters;
generating, using the processors, one or more predictive data representations for the input data object based on the predictive temporal parameters or the augmented temporal data objects;
generating, using the processors, a multi-channel predictive data representation based on the predictive data representations for the input data object; and
generating, using the processors and a machine-learning based input data object classification model, a predictive classification for the input data object based on the multi-channel predictive data representation.

2. The computer-implemented method of claim 1, wherein at least one predictive data representation of the predictive data representations is indicative of a heat map for at least one augmented temporal data object of the augmented temporal data objects.

3. The computer-implemented method of claim 2, wherein the heat map is a three-dimensional tensor comprising: (i) a first dimension indicative of a time associated with a predictive temporal parameter of the predictive temporal parameters, (ii) a second dimension indicative of a scale of the predictive temporal parameter, and (iii) a third dimension indicative of a degree to which the scale of the predictive temporal parameter is represented by the predictive temporal parameters.

4. The computer-implemented method of claim 3, wherein the predictive data representation comprises a predictive image comprising a plurality of image pixels, wherein an x-axis coordinate of an image pixel of the image pixels is indicative of the first dimension, a y-axis coordinate of the image pixel is indicative of the second dimension, and a color intensity of the image pixel is indicative of the third dimension.

5. The computer-implemented method of claim 3, wherein the third dimension is indicative of a wavelet transform coefficient of the first dimension and the second dimension.

6. The computer-implemented method of claim 1, wherein the machine-learning based input data object classification model is trained to generate the predictive classification based on the multi-channel predictive data representation.

7. The computer-implemented method of claim 6, wherein the machine-learning based input data object classification model comprises a convolutional neural network.

8. The computer-implemented method of claim 6, wherein the machine-learning based input data object classification model is trained using one or more machine-learning based techniques and based on a historical data object indicative of a plurality of historical predictive classifications and a plurality of historical predictive temporal parameters.

9. The computer-implemented method of claim 8, wherein the historical data object comprises a plurality of training pairs, wherein a training pair of the training pairs comprises a historical predictive classification and a historical multi-channel predictive data representation corresponding to a respective one of the historical predictive temporal parameters.

10. The computer-implemented method of claim 1, wherein the predictive temporal parameters comprise a plurality of sensor measurements over an evaluation time period.

11. The computer-implemented method of claim 10, wherein the sensor measurements are associated with a plurality of timestamps with respect to the evaluation time period, and wherein determining the augmented temporal data objects comprises:
generating, using the processors, a time-series model for the sensor measurements based on the timestamps; and
determining, using the processors, the augmented temporal data objects based on the time-series model.

12. An apparatus for machine-learning based input data augmentation and analysis, the apparatus comprising at least one processor and at least one memory including program code, the at least one memory and the program code configured to, upon execution by the at least one processor, cause the apparatus to:
determine one or more augmented temporal data objects based on a plurality of predictive temporal parameters of an input data object associated with a predictive classification process, wherein the augmented temporal data objects comprise at least one transformation of the predictive temporal parameters;
generate one or more predictive data representations for the input data object based on the predictive temporal parameters or the augmented temporal data objects;
generate a multi-channel predictive data representation based on the predictive data representations for the input data object; and
generate, using a machine-learning based input data object classification model, a predictive classification for the input data object based on the multi-channel predictive data representation.

13. The apparatus of claim 12, wherein at least one predictive data representation of the predictive data representations is indicative of a heat map for at least one augmented temporal data object of the augmented temporal data objects.

14. The apparatus of claim 13, wherein the heat map is a three-dimensional tensor comprising: (i) a first dimension indicative of a time associated with a predictive temporal parameter of the predictive temporal parameters, (ii) a second dimension indicative of a scale of the predictive temporal parameter, and (iii) a third dimension indicative of a degree to which the scale of the predictive temporal parameter is represented by the predictive temporal parameters.

15. The apparatus of claim 14, wherein the predictive data representation comprises a predictive image comprising a plurality of image pixels, wherein an x-axis coordinate of an image pixel of the image pixels is indicative of the first dimension, a y-axis coordinate of the image pixel is indicative of the second dimension, and a color intensity of the image pixel is indicative of the third dimension.

16. The apparatus of claim 14, wherein the third dimension is indicative of a wavelet transform coefficient of the first dimension and the second dimension.

17. A computer program product for machine-learning based input data augmentation and analysis, the computer program product comprising at least one non-transitory computer-readable storage medium having computer-readable program code portions stored therein, the computer-readable program code portions configured to:
determine one or more augmented temporal data objects based on a plurality of predictive temporal parameters of an input data object associated with a predictive classification process, wherein the augmented temporal data objects comprise at least one transformation of the predictive temporal parameters;
generate one or more predictive data representations for the input data object based on the predictive temporal parameters or the augmented temporal data objects;
generate a multi-channel predictive data representation based on the predictive data representations for the input data object; and
generate, using a machine-learning based input data object classification model, a predictive classification for the input data object based on the multi-channel predictive data representation.

18. The computer program product of claim 17, wherein the machine-learning based input data object classification model is trained to generate the predictive classification based on the multi-channel predictive data representation.

19. The computer program product of claim 18, wherein the machine-learning based input data object classification model comprises a convolutional neural network.

20. The computer program product of claim 18, wherein the machine-learning based input data object classification model is trained using one or more machine-learning based techniques and based on a historical data object indicative of a plurality of historical predictive classifications and a plurality of historical predictive temporal parameters.
